(19) United States
(12) Patent Application Publication
(10) Pub. No.: US 2026/0363852 A1
(43) Pub. Date: Nov. 19, 2026

(54) Prosthetic Atrioventricular Valve With Ventricular Anchors

(71) Applicant: St. Jude Medical, Cardiology Division, Inc., St. Paul, MN (US)

(72) Inventors: Beldanin Aslani Takieh, San Jose, CA (US); Dario Luppino, Mountain View, CA (US)

(73) Assignee: St. Jude Medical, Cardiology Division, Inc., St. Paul, MN (US)

(21) Appl. No.: 19/574,611

(22) Filed: Mar. 23, 2026

Related U.S. Application Data
(60) Provisional application No. 63/805,701, filed on May 14, 2025.

(51) Int. Cl. A61F 2/24

(52) U.S. Cl. CPC A61F 2/2418; A61F 2/2436; A61F 2220/0016; A61F 2250/0037

(57) ABSTRACT

A prosthetic atrioventricular valve includes a frame, prosthetic leaflets are mounted within the frame, and a plurality of anchor arms each having (i) a main body coupled to the frame and (ii) a tip at a free end of the anchor arm, the tip of the anchor arm having a width that is larger than a width of the main body. When the prosthetic valve is in an expanded condition, the tip of each anchor arm points toward the inflow end of the frame and is spaced radially outward of the frame to define a leaflet-receiving space. When the prosthetic valve is in the collapsed condition within a catheter of a delivery device, the tip of each anchor arm points away from the inflow end of the frame, and the tips of at least two of the anchor arms are positioned at different axial positions relative to the catheter.

### CROSS-REFERENCE TO RELATED APPLICATIONS

This application claims priority to U.S. Provisional Patent Application No. 63/805,701, filed May 14, 2025, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The human heart includes four heart valves, including the aortic valve, the mitral valve (also known as the bicuspid valve or the left atrioventricular valve), the pulmonary valve, and the tricuspid valve (also known as the right atrioventricular valve). Although prosthetic heart valves intended for use in replacing the various native heart valves may typically have design elements in common, each native heart valve has unique anatomical considerations that may need to be considered to design an optimal prosthetic heart valve. For example, the mitral valve and the tricuspid valve are typically significantly larger than the aortic valve and the pulmonary valve, and thus prosthetic heart valves designed to replace the mitral valve or the tricuspid valve typically need to have larger frames than those designed to replace the aortic valve or the pulmonary valve. Pressure generated by the left ventricle is typically significantly larger than pressure generated by the right ventricle, and thus prosthetic heart valves designed to replace the mitral valve typically need more substantial anchoring mechanisms than prosthetic heart valves designed to replace the tricuspid valve. While surgical prosthetic heart valves are typically sutured into place, transcatheter prosthetic heart valves typically need to rely on non-suture based anchoring, which may include frictional anchoring (e.g. via self-expansion forces or after balloon-expansion of a frame). While some prosthetic valves may rely solely on anchoring via radial expansion, such prosthetic valves are typically more suitable for use in the aortic valve or pulmonary valve due to lower forces that tend to displace the valve when the prosthetic leaflets are closed. As noted above, atrioventricular valves typically have relatively high forces that tend to display the prosthetic heart valve when the prosthetic leaflets are closed, and thus additional anchoring mechanisms may be necessary.

One example of additional anchoring mechanisms used in prosthetic transcatheter atrioventricular valves is one or more anchoring arms that hook around one or more native valve leaflets, or otherwise sandwich the one or more native valve leaflets between the one or more anchoring arms and a frame of the prosthetic heart valve. In these examples, the one or more anchoring arms help resist displacement of the prosthetic heart valve in a direction toward the atrium during ventricular systole. However, particularly when a plurality of anchor arms are used, it may be difficult to simultaneously place each anchor around a native leaflet successfully, and further the use of multiple anchor arms may tend to increase the size of the delivery device needed to deliver the prosthetic heart valve. Another potential issue with the use of such anchoring arms is the ability to readily and accurately confirm whether the anchor arms have been placed in their desired positions. Embodiments described herein may address one or more of these potential issues.

### BRIEF SUMMARY OF THE DISCLOSURE

According to one aspect of the disclosure, a system includes a prosthetic atrioventricular valve for implantation into a native heart valve, and a delivery device. The prosthetic atrioventricular valve includes a collapsible and expandable frame extending in a longitudinal direction from an inflow end of the frame toward an outflow end of the frame. Prosthetic leaflets are mounted within the frame and are configured to allow blood to flow through the prosthetic heart valve in an antegrade direction from the inflow end of the frame toward the outflow end of the frame, but to prevent blood from flowing through the prosthetic heart valve in a retrograde direction from the outflow end of the frame toward the inflow end of the frame. The prosthetic valve includes a plurality of anchor arms, each anchor arm having (i) a main body coupled to the frame and (ii) a tip at a free end of the anchor arm, the tip of the anchor arm having a width that is larger than a width of the main body. The delivery device includes a catheter configured to receive the prosthetic atrioventricular valve therein while the prosthetic atrioventricular valve is in a collapsed condition. When the prosthetic atrioventricular valve is in an expanded condition, the tip of each of the plurality of anchor arms points toward the inflow end of the frame and is spaced radially outward of the frame to define a leaflet-receiving space. When the prosthetic atrioventricular valve is in the collapsed condition within the catheter, the tip of each of the plurality of anchor arms points away from the inflow end of the frame, and the tips of at least two of the plurality of anchor arms are positioned at different axial positions relative to the catheter. When the prosthetic atrioventricular valve is in the collapsed condition within the catheter, no two circumferentially adjacent ones of the tips of the plurality of anchor arms may be positioned at a same axial position relative to the catheter. The plurality of anchor arms may include nine anchor arms arranged in a first group of three anchor arms, a second group of three anchor arms, and a third group of three anchor arms, such that when the prosthetic atrioventricular valve is in the collapsed condition within the catheter, the tips of the three anchor arms in the first group are positioned at a first axial position relative to the catheter, the tips of the three anchor arms in the second group are positioned at a second axial position relative to the catheter, and the tips of the three anchor arms in the third group are positioned at a third axial position relative to the catheter, the first axial position being different than the second axial position, the second axial position being different than the third axial position, and the third axial position being different than the first axial position. Each anchor arm in the first group may be positioned between one of the anchor arms in the second group and one of the anchor arms in the third group, and each anchor arm in the second group may be positioned between one of the anchor arms in the first group and one of the anchor arms in the third group, and each anchor arm in the third group may be positioned between one of the anchor arms in the first group and one of the anchor arms in the second group. The tips of the plurality of anchor arms may be formed with echogenic material.

According to another aspect of the disclosure, a system includes a prosthetic atrioventricular valve for implantation into a native heart valve and a delivery device. The prosthetic atrioventricular valve includes a collapsible and expandable frame extending in a longitudinal direction from an inflow end of the frame toward an outflow end of the frame. Prosthetic leaflets are mounted within the frame and are configured to allow blood to flow through the prosthetic heart valve in an antegrade direction from the inflow end of the frame toward the outflow end of the frame, but to prevent blood from flowing through the prosthetic heart valve in a retrograde direction from the outflow end of the frame toward the inflow end of the frame. The prosthetic valve includes a plurality of anchor arms, each anchor arm having (i) a main body coupled to the frame and (ii) a tip at a free end of the anchor arm. The delivery device includes a catheter configured to receive the prosthetic atrioventricular valve therein while the prosthetic atrioventricular valve is in a collapsed condition. When the prosthetic atrioventricular valve is in an expanded condition, the tip of each of the plurality of anchor arms points toward the inflow end of the frame and is spaced radially outward of the frame to define a leaflet-receiving space. When the prosthetic atrioventricular valve is in the collapsed condition within the catheter, the tip of each of the plurality of anchor arms points away from the inflow end of the frame, and the tips of at least two of the plurality of anchor arms are positioned at different axial positions relative to the catheter. The tips of the plurality of anchor arms are formed with echogenic material. The echogenic material may be a biocompatible metal or metal alloy. At least some of the plurality of anchor arms may include a frictional element on the main body adjacent to the tip. The frictional element may be selected from the group consisting of spikes, hooks, tines, or barbs. When the prosthetic atrioventricular valve is in the collapsed condition within the catheter, the tips of at least two of the plurality of anchor arms may be positioned at different axial positions relative to the catheter.

According to a further aspect of the disclosure, a method of implanting a prosthetic atrioventricular valve into a native atrioventricular valve of a patient includes positioning the prosthetic atrioventricular valve in a collapsed condition within a catheter of a delivery device. The prosthetic atrioventricular valve includes a collapsible and expandable frame extending in a longitudinal direction from an inflow end of the frame toward an outflow end of the frame, a plurality of prosthetic leaflets mounted within the frame, and a plurality of anchor arms, each anchor arm having (i) a main body coupled to the frame and (ii) a tip at a free end of the anchor arm, the tip of the anchor arm having a width that is larger than a width of the main body. The catheter is advanced through vasculature of the patient until the catheter is positioned in or adjacent to the native atrioventricular valve. During the advancing, the tip of each of the plurality of anchor arms points away from the inflow end of the frame, and the tips of at least two of the plurality of anchor arms are positioned at different axial positions relative to the catheter. When the catheter is positioned in or adjacent to the native atrioventricular valve, the catheter is withdrawn relative to the prosthetic atrioventricular valve so that a tip of a first one of the plurality of anchor arms exits the catheter before a tip of a second one of the plurality of anchor arms exits the catheter. At least one of the anchor arms is allowed to start to transition to a set shape in which the tip of the at least one of the anchor arms points toward the inflow end of the frame after the at least one of the anchor arms exits the catheter, so that the at least one of the anchor arms hooks behind a native leaflet of the native atrioventricular valve. After the at least one of the anchor arms hooks behind a native leaflet of the native atrioventricular valve, the prosthetic atrioventricular valve is fully deployed from the catheter and the prosthetic atrioventricular valve is allowed to self-expand into the native atrioventricular valve. When the prosthetic atrioventricular valve is in the collapsed condition within the catheter, no two circumferentially adjacent ones of the tips of the plurality of anchor arms may be positioned at a same axial position relative to the catheter. The plurality of anchor arms may include nine anchor arms arranged in a first group of three anchor arms, a second group of three anchor arms, and a third group of three anchor arms, such that when the prosthetic atrioventricular valve is in the collapsed condition within the catheter, the tips of the three anchor arms in the first group are positioned at a first axial position relative to the catheter, the tips of the three anchor arms in the second group are positioned at a second axial position relative to the catheter, and the tips of the three anchor arms in the third group are positioned at a third axial position relative to the catheter, the first axial position being different than the second axial position, the second axial position being different than the third axial position, and the third axial position being different than the first axial position. Each anchor arm in the first group may be positioned between one of the anchor arms in the second group and one of the anchor arms in the third group, each anchor arm in the second group may be positioned between one of the anchor arms in the first group and one of the anchor arms in the third group, and each anchor arm in the third group may be positioned between one of the anchor arms in the first group and one of the anchor arms in the second group. The tips of the plurality of anchor arms may be formed with echogenic material.

According to still another aspect of the disclosure, a method of implanting a prosthetic atrioventricular valve into a native atrioventricular valve of a patient includes positioning the prosthetic atrioventricular valve in a collapsed condition within a catheter of a delivery device. The prosthetic atrioventricular valve includes a collapsible and expandable frame extending in a longitudinal direction from an inflow end of the frame toward an outflow end of the frame, a plurality of prosthetic leaflets mounted within the frame, and a plurality of anchor arms, each anchor arm having (i) a main body coupled to the frame and (ii) a tip at a free end of the anchor arm, wherein the tips of the plurality of anchor arms are formed with echogenic material. The catheter is advanced through vasculature of the patient until the catheter is positioned in or adjacent to the native atrioventricular valve. During the advancing, the tip of each of the plurality of anchor arms points away from the inflow end of the frame. At least one of the anchor arms is allowed to start to transition to a set shape in which the tip of the at least one of the anchor arms points toward the inflow end of the frame after the at least one of the anchor arms exits the catheter, so that the at least one of the anchor arms hooks behind a native leaflet of the native atrioventricular valve. The tip of the at least one of the anchor arms is viewed under ultrasound imaging to determine that the tip of the at least one of the anchor arms has a desired position relative to the native leaflet of the native atrioventricular valve. After confirming that the tip of the at least one of the anchor arms has the desired position relative to the native leaflet of the native atrioventricular valve, the prosthetic atrioventricular valve is fully deployed from the catheter and the prosthetic atrioventricular valve is allowed to self-expand into the native atrioventricular valve. The echogenic material may be a biocompatible metal or metal alloy. At least some of the plurality of anchor arms may include a frictional element on the main body adjacent to the tip, and when the at least one of the anchor arms hooks behind the native leaflet of the native atrioventricular valve, the frictional element may frictionally engage tissue of the native leaflet. The frictional element may be selected from the group consisting of spikes, hooks, tines, or barbs. When the prosthetic atrioventricular valve is in the collapsed condition within the catheter, the tips of at least two of the plurality of anchor arms may be positioned at different axial positions relative to the catheter.

### BRIEF DESCRIPTION OF THE DRAWINGS

FIG. 1 is a perspective view of a prosthetic atrioventricular valve according to an aspect of the disclosure.

FIG. 2A is a schematic cross-section of the prosthetic atrioventricular valve of FIG. 1.

FIG. 2B is a schematic cross-section of an alternative example of the prosthetic atrioventricular valve of FIG. 1.

FIG. 3 shows the prosthetic heart valve of FIGS. 1-2A in an implanted condition.

FIGS. 4A-4C show example steps of a portion of sequence of deploying anchor arms of the prosthetic heart valve of FIGS. 1-2A from a catheter of a delivery device.

FIG. 5 is a perspective view of a prosthetic atrioventricular valve according to another aspect of the disclosure having anchor arms of staggered lengths.

FIG. 6 is a schematic view of the frame of the prosthetic heart valve of FIG. 5 as if cut longitudinally and laid out flat on a table, with all of the anchor arms being shown in an inverted condition they would have during delivery.

FIG. 7 is a perspective view of a prosthetic atrioventricular valve according to another aspect of the disclosure having echogenic anchor arms with frictional elements.

FIGS. 8A-8C illustrate examples of anchor arms having different spring tensions.

FIGS. 9A-9C illustrate examples of anchor arms having different set angles shown from a side or sectional view.

FIGS. 10A-10C illustrate examples of anchor arms having different set angles shown from a front view.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure may apply with similar force to prosthetic mitral valves. Further, although the features disclosed herein may be particularly useful in prosthetic atrioventricular valves, the features may still be applied to any transcatheter prosthetic heart valve replacements. Thus, unless otherwise expressly specified, the embodiments described herein may be used for replacing any native heart valve (with or without additional modifications specific to the heart valve being replaced), even if a particular embodiment may be more suited for replacing the native tricuspid valve than another native heart valve.

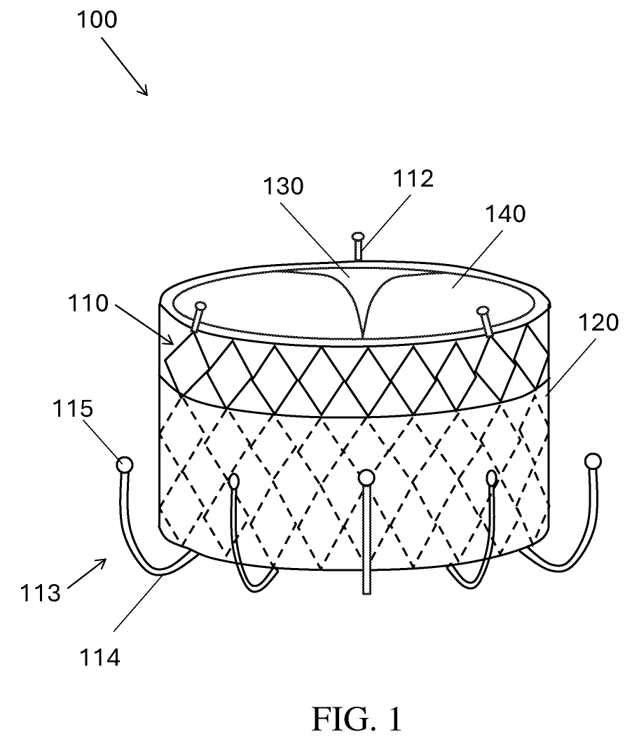

FIG. 1 is a perspective view of a prosthetic heart valve 100, in particular a prosthetic atrioventricular valve, shown in an expanded condition. Prosthetic heart valve 100 may include a collapsible and expandable frame 110. Frame 110 may be formed of a biocompatible shape memory or superelastic material. One suitable example of this frame material is a nickel-titanium alloy, such as nitinol. However, other materials may be suitable. In one example, frame 110 may be formed by laser cutting a hollow tube of nitinol, and then shape-setting the frame 110 to the desired shape, for example by heat treatment. With this configuration, the frame 110 may take the set shape, such as that shown in FIG. 1, in the absence of applied forces. To deliver the prosthetic heart valve 100, the prosthetic heart valve 100 may be collapsed to a small diameter and positioned within a delivery catheter to be passed intravascularly through the patient into the patient’s heart.

In the example of FIG. 1, the frame 110 is generally cylindrical in the expanded or shape-set condition, and includes a plurality of annular rows of cells extending around the circumference of the frame 110. In the illustrated example, each cell is generally diamond-shaped, but it should be understood that other numbers of rows of cells than shown, and other shapes of cells than shown, may be appropriate in place of generally diamond-shaped cells. Further, although the frame 110 is shown as being shape-set to a cylindrical shape, other shapes may be suitable. For example, the inflow end of the frame 110 (at the top in the view of FIG. 1) may be flared radially outwardly, or tapered radially inwardly. Similarly, the outflow end of the frame 110 (at the bottom in the view of FIG. 1) may be flared radially outwardly, or tapered radially inwardly. In some examples, each annular row of cells include a total number of cells that is a whole-number multiple of three, including for examples three cells, six cells, nine cells, or twelve cells. However, other numbers of cells may be suitable.

One or more valve retainers 112 may be provided on the frame 110, for example at or near the inflow end of the frame. In some examples, each valve retainer 112 is integrally formed with the frame 110 and extends in the inflow direction from an inflow apex of a cell in the inflow-most row of cells. Each valve retainer 112 may include a post or a shaft that terminates in an enlarged head (which may have a circumferential width that is larger than the circumferential width of the post or shaft). If included, the valve retainers 112 may engage with a corresponding retainer on a delivery device to help ensure the prosthetic heart valve 100 remains engaged with the delivery device (e.g. while still allowing the prosthetic heart valve 100 to be re-sheathed back into the delivery device) until the physician is ready to fully release the prosthetic heart valve 100 from the delivery system. For example, the enlarged heads of the valve retainers 112 may be received within a correspondingly-shaped recess in the retainer on the delivery device, and the shafts of the valve retainers 112 may be received within a correspondingly-shaped channel in the retainer on the delivery device, such that, as long as an overlying sheath of the delivery device remains positioned over the valve retainers 112, the prosthetic heart valve 100 is prevented from fully releasing from the prosthetic heart valve 100. In some examples, the prosthetic heart valve 100 includes three retainers 112 spaced at about equal intervales around the circumference of the frame, but other numbers of retainers 112, including for example six, nine, or twelve, may also be suitable.

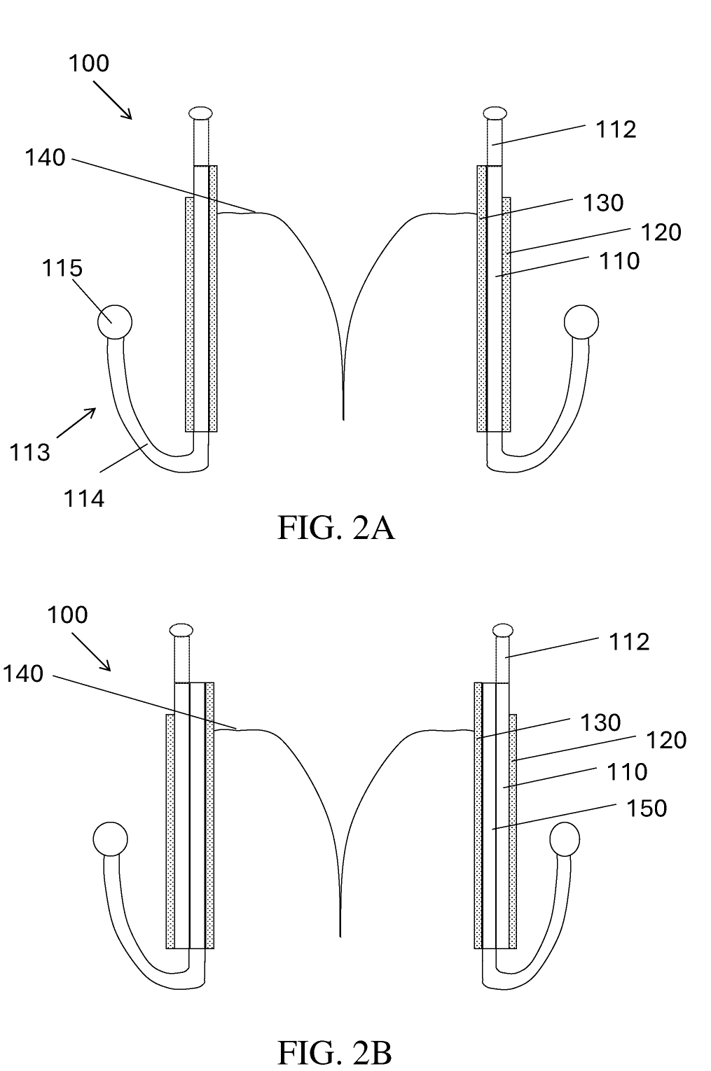

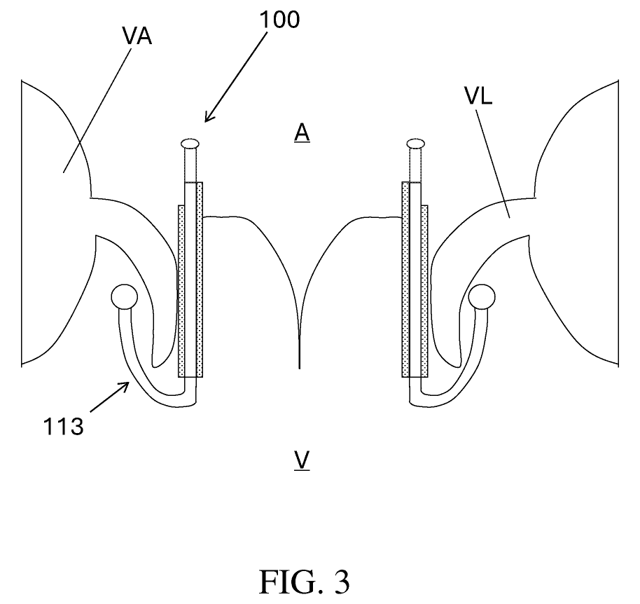

Referring to FIGS. 1 and 2A, prosthetic heart valve 100 may also include a plurality of anchor arms 113 which may be configured to hook around the back of the native heart valve leaflets to assist with anchoring the prosthetic heart valve 100, for example by helping resist displacement of the prosthetic heart valve 100 into the atrium during ventricular systole. Each anchor arm 113 may have a first end coupled to (or adjacent to) an outflow end of the frame 110. In some examples, the anchor arms 113 (or at least a portion thereof) may be formed integrally with the frame 110, although in other examples the anchor arms 113 may be formed separately from the frame 110 and then later attached to the frame 110. Each anchor arm 113 may have a main body 114, a first end of the main body being attached to the frame 110, a second free end of the main body 114 terminating in a tip 115. In some examples, the tip 115 may take the form of an enlarged head. For example, the tip 115 may be formed by adding padding or other material to the end of the main body 114 to create a more atraumatic end which is less likely to damage the native heart valve tissue. In some examples, the tip 115 may be generally spherical or ball-shaped, and in other examples the tip 115 may be generally cylindrical (or otherwise in the form of a generally flat circle). In the expanded condition shown in FIGS. 1-2A, the arms 113 may extend radially outward from the frame 110 (sometimes with an initial extension in the outflow direction before extending back in the inflow direction) so that the tip 115 and at least some portion of the arm 113 extends generally in the inflow direction at a spaced distance radially outward from the outer surface of the frame 110. With this configuration, when in the expanded condition, the prosthetic heart valve 100 may receive portions of the native heart valve leaflets between the anchor arms 113 and the radially outer surface of the frame 110. In other words, the anchor arms 113 may be shaped, in the expanded condition, to hook behind the native heart valve leaflets.

The prosthetic heart valve 100 may include any suitable number of anchor arms 113. For example, the prosthetic heart valve 100 may include two, three, four, five, six, nine, twelve, or more anchor arms 113. In some examples, the frame 110 includes a number of cells in each annular row that is a whole number multiple of three, and a whole number multiple of three anchor arms 113 are provided. For example, a frame 110 with nine cells per row may include three, six, or nine anchor arms 113, preferably at substantially equal intervals around the frame 110. If the frame 110 includes rows of nine cells, and nine anchor arms 113, each anchor arm 113 may extend from a corresponding cell in the outflow-most row of cells. Further, in the expanded condition, the tip 115 of each anchor arm 113 may be spaced at any desirable distance in the axial direction between the inflow and outflow ends of the frame. For example, the tip 115 may be positioned at about the halfway point in the axial direction between the inflow and outflow ends of the frame 110, about 25% of the distance (either closer to the inflow end or to the outflow end), or about 33% of the distance (either closer to the inflow end or to the outflow end).

As is described in greater detail below, when the prosthetic heart valve 100 is maintained in the collapsed condition within a sheath of a delivery device, the anchor arms 113 may be inverted compared to their orientation shown in FIGS. 1-2A, for example with the main body 114 being relatively straight or otherwise aligned with the frame 100 and extending completely in the outflow direction from the outflow end of the frame 110. With this configuration, the tips 115 may be the first portion of the prosthetic heart valve 100 exposed when the delivery sheath is withdrawn, and the anchor arms 113 may begin to flip or revert in orientation (back toward the shape-set condition shown in FIGS. 1-2A), with the anchor arms 113 flipping behind the native heart valve leaflets during deployment. One benefit of this configuration is that the frame 110 and the arms 113 do not radially overlap while maintained in the collapsed condition within the delivery sheath, reducing the total bulk and thus the size that the delivery sheath needs to be to contain the prosthetic heart valve 100.

Prosthetic heart valve 100 may include one or mor cuffs or skirts that may function to assist with sealing the prosthetic heart valve 100 from blood flowing through the cells of the frame 110 and/or to assist with sealing against the native tissue so that blood does not flow around the outside of the prosthetic heart valve (also known as paravalvular or “PV” leak). In one example, the prosthetic heart valve includes an exterior skirt 120 and an interior skirt 130, although in other examples a single skirt (either exterior or interior) may be provided. The outer skirt 120 and/or inner skirt 130 may be formed of any suitable biocompatible material, including tissues (such as bovine or porcine pericardial tissue) or fabrics (including synthetic fabrics such as polyethylene terephthalate (“PET”), polytetrafluoroethylene (“PTFE”), or other polymer fabrics). In the illustrated example, the outer skirt 120 extends from at or near the outflow end of the frame 110 toward the inflow end of the frame 110, but not the entire axial distance (e.g. about 50%, about 66%, about 75%, etc.). In other examples, the outer skirt 120 may extend the entire axial distance of the frame 110. While the outer skirt 120 extends around the abluminal surface of the frame 110, the inner skirt 130 extends around the luminal surface of the frame 110. In the illustrated example, the inner skirt 130 extends from at or near the outflow end of the frame 110 to (or near to) the inflow end of the frame 110. However, if included, the inner skirt 130 may extend other axial distances less than the entire axial distance of the frame.

Referring still to FIGS. 1-2A, the prosthetic heart valve 100 may include two or more prosthetic leaflets 140. In the illustrated example, prosthetic heart valve 100 includes three prosthetic leaflets 140. The prosthetic leaflets 140 may be formed of any suitable biocompatible material, including any of those described above in connection with the outer skirt 120 and inner skirt 130. The prosthetic leaflets 140 generally create the valve functionality, with the prosthetic leaflets 140 moving away from each other to allow blood to flow through the prosthetic heart valve 100 during atrial systole, and moving together toward each other to coapt during ventricular systole to prevent blood from flowing in the retrograde direction through the prosthetic heart valve 100. In some examples, each leaflet 140 may have a generally “U”-shaped belly portion (a top portion of which is visible for two leaflets 140 in FIG. 1) that is sutured or otherwise attached to the frame 110 (such as by suturing to inner skirt 130, with the inner skirt sutured to the frame 110 to minimize or eliminate direct suturing of the bellies of the leaflets to the frame 110). Each leaflet 140 may also have a free edge portion which is not directly attached to the frame 110, with the free edges being the portions that coapt with each other (e.g. the bottom of the two leaflets 140 in the view of FIG. 2A). The leaflets 140 may also include tabs, with tabs of adjacent leaflets 140 being attached to each other to form prosthetic commissures, which may be attached directly or indirectly to the frame 110. It should be understood that many variations are possible for the design and implementation of prosthetic leaflets, and the above-described features of prosthetic leaflets 140 are just one example.

Although prosthetic heart valve 100 is shown and described as having a single frame 110, in other examples, the prosthetic heart valve 100 may include two frames, with frame 110 being an outer frame, with an inner frame 150 positioned inward of (and coupled to) the outer frame 110, for example as shown in the alternate example of FIG. 2B. In the example of FIG. 2B, the prosthetic heart valve 100 includes anchor arms 113 that may be substantially identical to those described in connection with FIG. 2A, except that the anchor arms 113 are attached to an inner frame 150 (which may be generally similar or identical to frame 110 described above). The frame 110 of prosthetic heart valve 100 of FIG. 2B may be identical to frame 110 described in connection with FIG. 2A, except that the frame is an outer frame 110 positioned around the outside of inner frame 150. In some examples, the inner frame 150 and outer frame 110 may have generally the same structure with generally similar or identical cells that align with each other when the outer frame 110 overlaps the inner frame 150. The outer frame 110 may be coupled to the inner frame 150, for example via suturing. The valve retainers 112, if included, may be positioned on the outer frame 110, the inner frame 150, or both. In the illustrated example, inner skirt 130 is provided on the luminal surface of inner frame 150, and outer skirt 120 is provided on the abluminal surface of outer frame 110. In some examples, if both an inner frame 150 and outer frame 110 are included, a third skirt or other buffer material may be provided between the two frames to prevent metal-on-metal contact, if desired. Features of other prosthetic atrioventricular valves with anchor arms are shown and described in U.S. patent Application Publication No. 2025/0107889, the disclosure of which is hereby incorporated by reference herein.

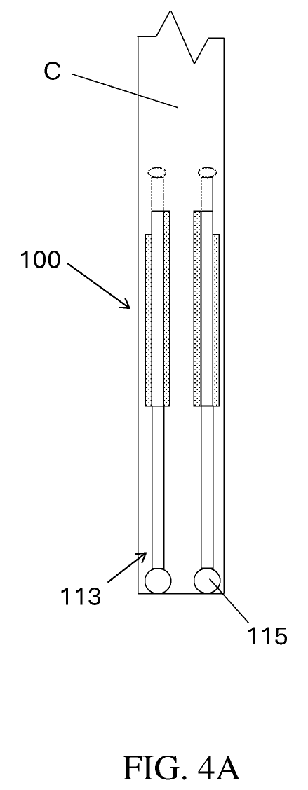

FIG. 3 is a highly schematic view of the prosthetic heart valve 100 of FIGS. 1-2A in an implanted condition within a native heart valve which is shown in simplified form. As shown in FIG. 3, the prosthetic heart valve 100 is implanted within the native valve annulus VA so that the inflow end of the prosthetic heart valve 100 is positioned nearer the atrium A, the outflow end of the prosthetic heart valve 100 is positioned nearer the ventricle V, with the anchor arms 113 hooking behind the native valve leaflets VL. It should be understood that the native heart valve shown in FIG. 3 could be a tricuspid valve (right atrioventricular valve) or mitral valve (left atrioventricular valve). However, as described above, in other situations the prosthetic heart valve 100 may be configured for implantation into a native aortic or pulmonary valve.

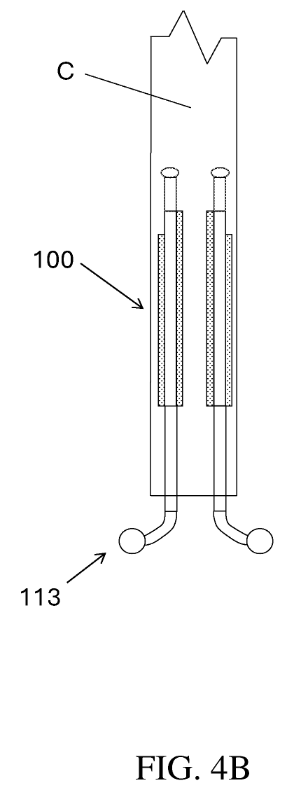

In one example, the anchor arms 113 of prosthetic heart valve may be similar or identical to each other in configuration, including length and set-shape. In that example, as the prosthetic heart valve 100 is deployed from a catheter C of a delivery device, each anchor arm 113 deploys at substantially the same time and begins to revert from the inverted shape to the set shape at about the same time. An example of a portion of the sequence in which the anchor arms 113 are deployed from a distal end of a delivery catheter C is shown in FIGS. 4A-4C, whereby the two illustrated anchor arms 113 have the same length (including tips 115 being aligned at the same position along the length of the catheter C). At least two potential disadvantages may result from all of the anchor arms being substantially identically configured and substantially the same length. First, if all of the tips 115 are at the same position in the axial direction in the collapsed delivery condition, all of the tips 115 will exit the catheter C simultaneously and will begin to revert to their set shape simultaneously. As a result of this, the physician must try to hook every anchor arm 113 around the native valve leaflets simultaneously. This may be a difficult task, particularly when there are a large number of anchor arms 113, such as six or nine arms total. Second, if all of the tips 115 are at the same position in the axial direction in the collapsed delivery condition, all of the tips 115 will occupy the same cross-section of the catheter C. In other words, the inner diameter of the catheter C must be large enough to accommodate all of the tips 115 at the same location, which may increase the size that the catheter C needs to be. It is generally desirable for the delivery catheter C to be as small as possible to aid in passing the delivery catheter C through the vasculature.

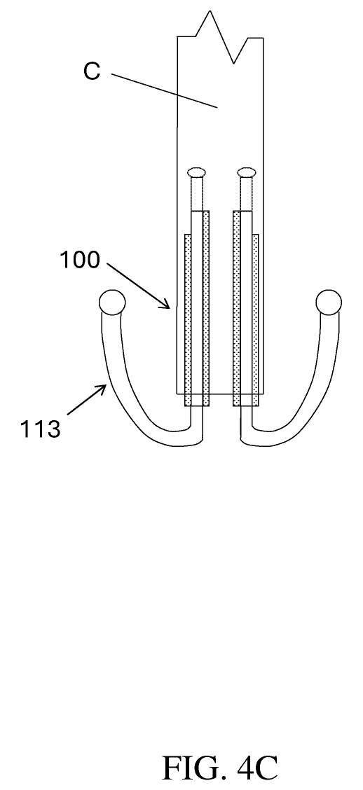

FIG. 5 is a perspective view of a prosthetic heart valve 200 which may be similar or identical to prosthetic heart valve 100 (including the versions shown and described in connection with FIGS. 1-2A and FIG. 3, as well as the alternative version shown and described in connection with FIG. 2B, except for the configuration of the anchor arms. For example, prosthetic heart valve 200 may include a frame 210, retainers 212, an outer cuff 220, an inner cuff 230, and valve leaflets 240 that may be similar or identical to their counterpart components in prosthetic heart valve 100 (including the option of a second frame). Prosthetic heart valve 200 may also include anchor arms, including in the number and/or relative positions described in connection with the anchor arms 113 of prosthetic heart valve 100. However, unlike anchor arms 113, the anchor arms of prosthetic heart valve 200 may be configured to have different lengths, or at least for the tips of the anchor arms to occupy different axial positions, particularly when the prosthetic heart valve 200 is in a collapsed delivery condition.

In one example, shown in FIG. 5, the prosthetic heart valve 200 may include a total of nine anchor arms, which are provided in groups having different lengths (or different axial positions of the anchor arm tips). With the example of nine anchor arms, the anchor arms may be provided in groups of three, including a group of first anchor arms 213a, a group of second anchor arms 213b, and a group of third anchor arms 213c. In the illustrated example, the only difference between the different groups of the anchor arms 213a-213c is the length of their main bodies 214a-c. For example, main bodies 214a may have a greater length than main bodies 214b, which may have a greater length than main bodies 214c. Each of the main bodies 214a-214c may terminate in a corresponding tip 215a-215c, which may (but need not) all be identical. Although the different length anchor arms may be positioned with various different configurations, in some examples it is preferred that no two immediately adjacent anchor arms have the same length.

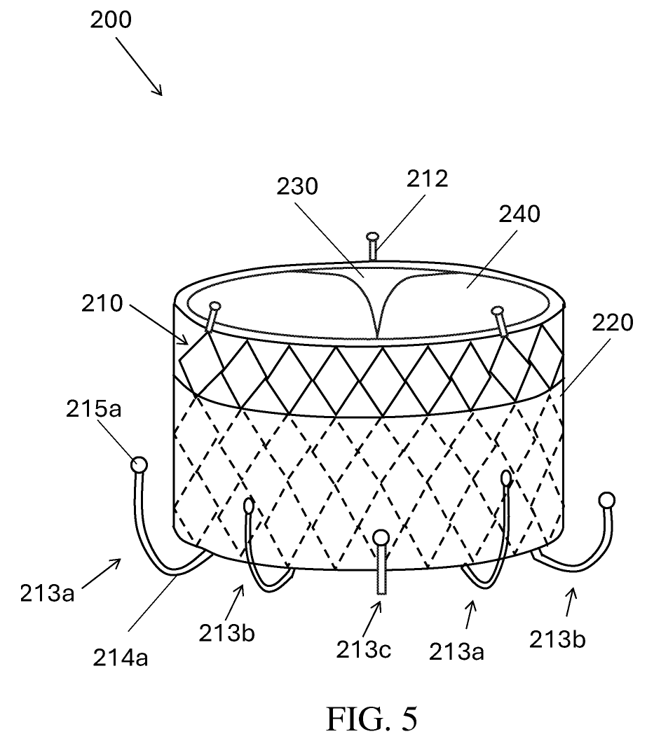

FIG. 6 is a schematic view of frame 210 of prosthetic heart valve 200 as if cut longitudinally and laid out flat on a table, with all of the anchor arms being shown in the inverted condition they would have during delivery (similar to the delivery configuration shown in FIG. 4A). In this example, each mid-length anchor arm 213b is positioned between a long-length anchor arm 213a and a short-length anchor arm 213c. Similarly, each short-length anchor arm 213c is positioned between a long-length anchor arm 213a and a medium-length anchor arm 213b. Finally, each long-length anchor arm 213a is positioned between a medium-length anchor arm 213b and a short-length anchor arm 213c.

With the configuration shown in FIG. 6, when the prosthetic heart valve is in a collapsed delivery condition within a catheter C of delivery device similar to that shown in FIG. 4A, the enlarged tips 215a-215c of the anchor arms 213a-213c will have a staggered configuration in which only three tips occupy the same axial length along the catheter C, instead of all nine tips occupying the same axial length as occurs with prosthetic heart valve 100. In other words, three tips 215a will be positioned at a first axial location within the catheter C, three tips 215b will be positioned at a second axial location within the catheter C that is different than the first axial location, and three tips 215c will be positioned at a third axial location within the catheter that is different than the first and second axial locations. By staggering the axial positions of the tips 215a-215c, particularly if the tips 215a-215c have large widths and/or large spherical shapes, the inner diameter of the catheter C may be smaller than might be possible if all nine tips were positioned at the same axial location within the catheter, allowing the outer diameter to be correspondingly smaller. As noted above, it is typically desirable for delivery catheters such as catheter C to have the smallest outer diameter possible while still suitably accommodating the prosthetic heart valve 200 therein for delivery.

Still referring to FIGS. 5-6, as the prosthetic heart valve 200 is deployed from the catheter C of the delivery device, similar as to the sequence shown in FIGS. 4A-4C, the three groups of anchor arms 213a-213c exit the distal end of the catheter C in different stages. In other words, the three anchors 213a will first exit the catheter C during deployment and begin to revert to their set shape (similar to as shown in FIGS. 4A-4C). As this occurs, the physician (or other user) may first capture the native valve leaflets VL with the tips 215a of anchor arms 213a before the anchor arms 213b and 213c are deployed (or fully deployed) from the catheter C. Then, as deployment continues, the physician (or other user) may next capture the native valve leaflets VL with the tips 215b of anchor arms 213b before the anchor arms 213c are deployed (or fully deployed) from the catheter C. Finally, the physician (or other user) may then capture the native valve leaflets VL with the tips 215c of anchor arms 213c, before the remainder of the prosthetic heart valve 200 is deployed from the catheter C. With this methodology, the user does not need to try to simultaneously seat all nine anchor arms to capture the native valve leaflets VL, but instead can focus on a fewer number of anchors at any one time due to the staggered release of the anchors 213a-213c from the catheter C. In some examples, after the first set of anchors 213a are hooked around the valve leaflets VL, the remaining anchors 213b, 213c may be easier to position because the valve leaflets VL have already been “grabbed” by the first set of anchors 213a.

It should be understood that more or fewer than nine anchor arms may be provided, and the anchor arms may be provided in fewer or more groups of differing lengths (or at least differing axial positions of the tips). For example, as few as two groups of different anchor arm lengths may be provided (e.g. alternating lengths, particularly if an even number of anchor arms are provided), or as many groups as the total number of anchor arms (e.g. every anchor arm has a unique length) may be provided. Further, although the difference in positions of the anchor arm tips is described as being achieved by varying the lengths of the main bodies 214, in some examples, the main bodies 214 may all be the same length, but they may be attached to the frame at different axial positions, resulting in the tips 215 having the desired different axial positions.

As noted above, successfully positioning anchor arms of a prosthetic heart valve behind native valve leaflets VL in a desired position may be difficult, particularly given that the procedure is being performed in a minimally invasive manner in which there is no possibility of direct line-of-sight visualization of the anchor arms deploying and engaging the native valve leaflets VL.

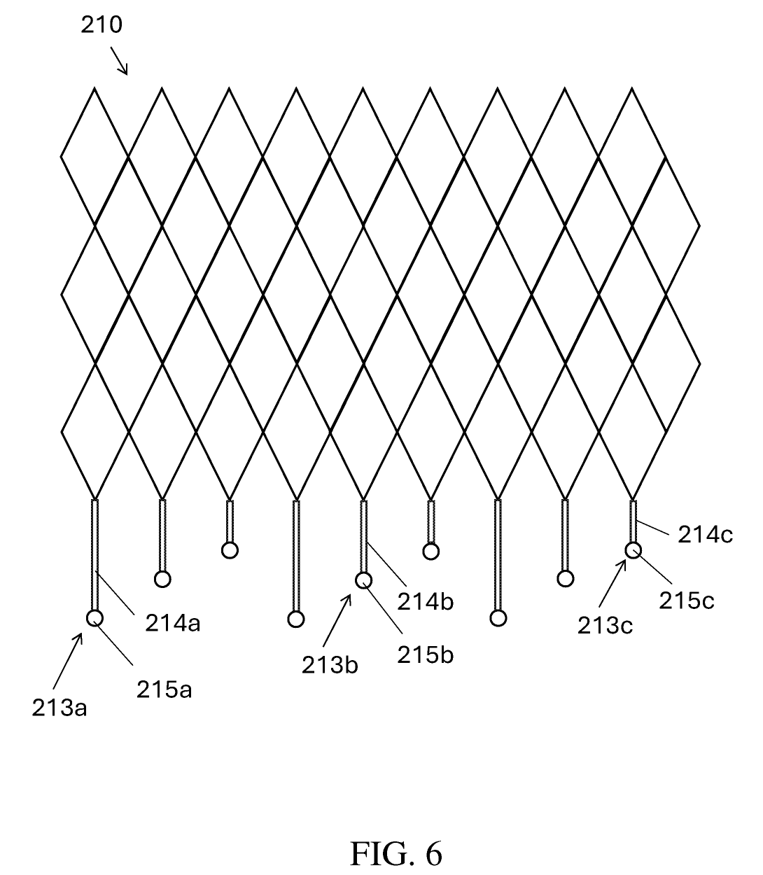

FIG. 7 is a perspective view of a prosthetic heart valve 300 which may be similar or identical to prosthetic heart valves 100 and 200 (including the alternative version shown and described in connection with FIG. 2B), except for one or more additional features provided with the anchor arms. For example, prosthetic heart valve 300 may include a frame 310, retainers 312, an outer cuff 320, an inner cuff 330, and valve leaflets 340 that may be similar or identical to their counterpart components in prosthetic heart valves 100 and 200 (including the option of a second frame). Prosthetic heart valve 300 may also include anchor arms, including in the number and/or relative positions described in connection with the anchor arms 113 of prosthetic heart valve 100 or in connection with the anchor arms 213a-213c of prosthetic heart valve 200 (as well as the alternatives described therewith). However, unlike anchor arms 113 and 213, the anchor arms 313 of prosthetic heart valve 300 include one or more frictional elements 316 and/or echogenic characteristics. It should be understood that although both the frictional elements 316 and the echogenicity are both described in connection with prosthetic heart valve 300, either feature may be used without the other, and either or both features may be used in combination with the features of prosthetic heart valve 200.

As shown in FIG. 7, each anchor arm 313 may be generally similar to anchor arms 113 and 213, and may include a main body 314 with a first end coupled to the frame 310 and a second free end terminating in a tip 315, which may be an atraumatic tip and which may include a feature such as a ball or sphere shape or which may otherwise have at least one dimension that is wider than the width of the main body 314. In FIG. 7, the tip 315 of each anchor arm 313 is shown with an “x” to indicate that the tip 315 is formed of (and/or covered with) a material that is readily visible under ultrasound imaging (e.g. sonography such as echocardiography). Such echogenic or echo-friendly materials may include, for example, biocompatible metals and metal alloys. By forming the anchor arm tips 315 (or material on or covering the tips 315) from an echogenic material, the physician may more readily assess the position of anchor arms 313 during deployment. For example, referring back to FIG. 3, if performing the implantation under ultrasound imaging, the tips 315 of the anchor arms 313 will be readily visible on the imaging display. By having the tips 315 of the anchor arms 313 readily visible under imaging during deployment, the position of each tip 315 may be viewed relative to the native valve leaflets VL to help the physician determine the amount that each anchor arm 313 (and particularly the tip 315) is being inserted relative to the valve leaflets VL, as well as the position of each anchor arm 313 (and particularly the tip 315) relative to the valve leaflets VL. Thus, the physician can determine whether each anchor arm 313 is desirably engaged with a native valve leaflet VL prior to fully deploying the prosthetic heart valve 300 and fully releasing the prosthetic heart valve 300 from the catheter. As used herein, the term “desirably engaged” may refer to (i) engagement of the anchor arm 313 with the native valve leaflet VL, (ii) the amount or distance which the anchor arm 313 is inserted relative to the native valve leaflet VL, and (iii) the general orientation of the anchor arm 313 relative to the native valve leaflet VL and any surrounding tissue.

After the physician has determined, by assessing the position of the echogenic anchor arm tips 315 under ultrasound imaging, that the anchor arms 313 are in satisfactory positions, the user may continue to deploy the prosthetic heart valve 300 from the catheter C of the delivery device, allowing for full engagement of the prosthetic heart valve 300 with the native valve leaflets VL. This full engagement may also include the frictional elements 316 frictionally engaging the native valve leaflets VL. In some examples, one or more (including all) of the anchor arms 316 include at least one frictional element 316, which may take the form of a spike, a tine, a hook, a barb, or any other feature configured to enhance frictional engagement. In some examples, the at least one frictional element 316 may be provided on the main body 314 adjacent to the tip 315, oriented so that, in the deployed condition of the prosthetic heart valve 300, the frictional elements 316 are generally oriented toward the axial center of the prosthetic heart valve 300 so that the frictional elements 316 engage the underside of the native valve leaflets VL. In some examples, after engaging the frictional elements 316 with the native valve leaflets VL (which may include prior to complete release of the prosthetic heart valve 300 from the catheter C of the delivery device while re-sheathing is still possible), the user may assess the engagement under imaging such as ultrasound imaging. For example, the movement of the native valve leaflets VL (or the lack thereof) may be viewed on the imaging screen, and if the movement of the native valve leaflets VL has significantly reduced or completely stop, the user may determine or confirm that the prosthetic heart valve 300 has achieved good securement. If this occurs prior to full release of the prosthetic heart valve 300, the prosthetic heart valve 300 may be fully deployed after such confirmation is achieved. As mentioned above, it should be understood that a prosthetic heart valve may include the frictional elements 316 even if the echogenic material is not provided for the tips 315, or otherwise the frictional elements 316 may be omitted when echogenic material is provided for the tips 315. Furthermore, either or both of these features may be used in conjunction with the staggered anchor arms described in connection with prosthetic heart valve 200.

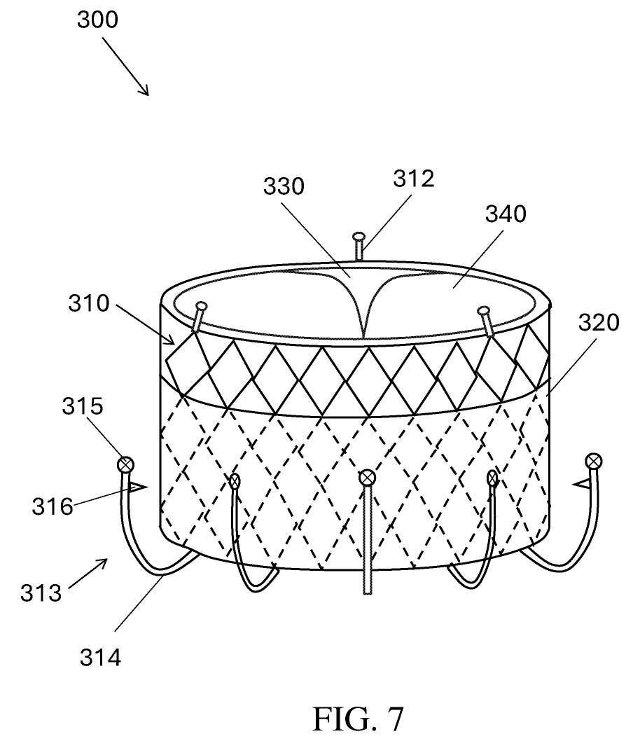

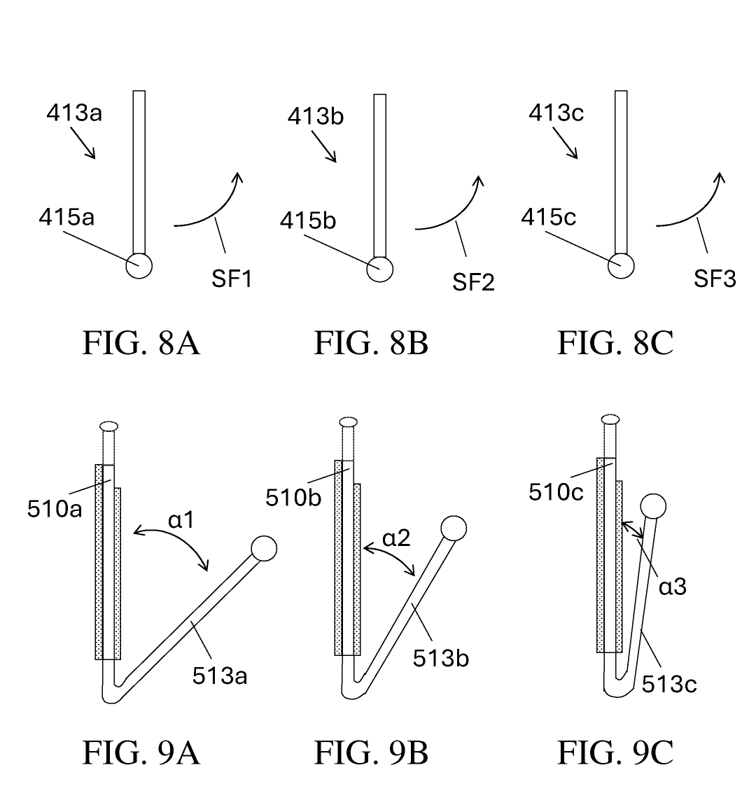

FIGS. 8A-8C illustrate examples of anchor arms 413a-413c (with the remainder of the prosthetic heart valve omitted from the view) in inverted delivery conditions similar to that shown in FIG. 4A, with the delivery catheter omitted from the view for clarity. It should be understood that the anchors arms 413a-413c may be used in conjunction with any other embodiment described herein. Anchor arms 413a-413c may each terminate in a tip 415a-415c similar or identical to any of the other anchor arms described herein. Anchor arms 413a-413c may be configured so that, when the prosthetic heart valve is in the delivery condition with the anchor arms inverted in the condition shown in FIGS. 8A-8C, the anchor arms 413a-413c have different levels of spring tension or force SF1-SF3. In other words, anchor arm 413a may have a relatively large spring tension SF1, anchor arm 413c may have a relatively small spring tension SF3, and anchor arm 413b may have an intermediate spring tension SF2 that is smaller than spring tension SF1 but larger than spring tension SF3. With this configuration, as the delivery catheter is withdrawn, anchor arm 413a may tend to revert back to its set shape relatively quickly due to the high spring force SF1, while anchor arm 413c may tend to revert back to its set shape relatively slowly due to the low spring force SF3, with the anchor arm 413b reverting back to its set shape at an intermediate speed due to its intermediate spring force SF2. In use, a prosthetic heart valve having anchor arms that are the same size (e.g. similar to prosthetic heart valve 100 or 300), or a prosthetic heart valve having anchor arms that are grouped in two or more different sizes (e.g. similar to prosthetic heart valve 200), may incorporate two or more groups of anchor arms with different spring tensions. For example, prosthetic heart valve 100 or 300 may be provided with a first group of three anchor arms 413a, a second group of three anchor arms 413b, and a third group of three anchor arms 413c, which may be provided in alternating positions around the circumference of the prosthesis. With this example, as the delivery catheter is withdrawn, anchor arms 413a may “spring” back to their set shape first, trailed by anchor arms 413b and then anchor arms 413c, due to the differential spring force SF1-SF3. This may assist with more easily placing the anchor arms 413a-413c without needing to simultaneously place all nine anchor arms in a desired position. It should be understood that anchor arms with only two different spring forces, or more than three different spring forces, may be used. It should also be understood that the different spring forces may be applied to anchor arms having different lengths, for example longer anchor arms having a larger spring force and shorter anchor arms having a smaller spring force (or vice versa). In some examples, the engagement arms may be formed with different spring tensions by shape-setting the arms (e.g. via heat treatment) to have different set angles (e.g. as described below in connection with FIGS. 9A-C), or by varying the material and/or geometry (e.g. width and/or thickness) of the engagement arms.

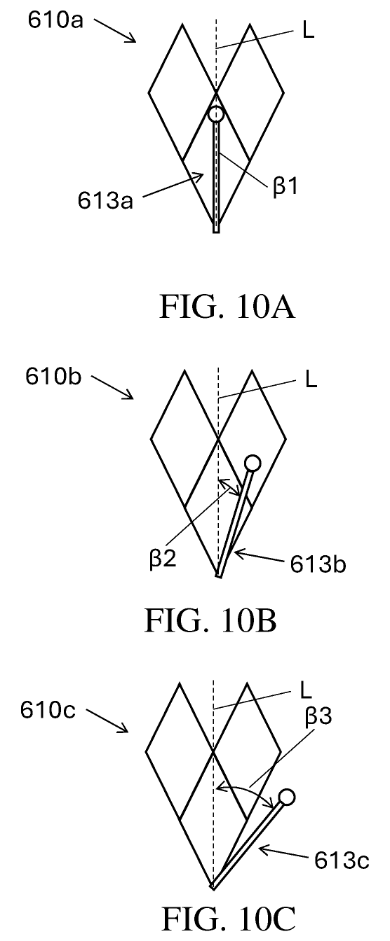

FIGS. 9A-9C illustrate more examples of prosthetic heart valves, with FIGS. 9A-9C showing portions of frames 510a-510c (inner and outer skirts are also shown, but not labeled, in FIGS. 9A-9C). The prosthetic heart valves shown in FIGS. 9A-9C may be substantially similar or identical to the other prosthetic heart valves described herein, with the primary difference of the embodiments of FIGS. 9A-9C being the different set angles α1, α2, α3 of the engagement arms 513a-513c. The views of FIGS. 9A-9C illustrate a portion of the frame 510a-510c when in an expanded or deployed condition, or otherwise in the absence of applied forces. It should be understood that the prosthetic heart valves represented in FIGS. 9A-9C may include any number of engagement arms 513a-513c, similar to other embodiments described herein. As shown in FIG. 9A, in the absence of applied forces, engagement arm 513a forms an angle of α1 between the engagement arm 513a and a longitudinal axis of the prosthetic heart valve (and/or with an outer surface of the frame 510a). As shown in FIG. 9B, in the absence of applied forces, engagement arm 513b forms an angle of α2 between the engagement arm 513b and a longitudinal axis of the prosthetic heart valve (and/or with an outer surface of the frame 510b). As shown in FIG. 9C, in the absence of applied forces, engagement arm 513c forms an angle of α3 between the engagement arm 513c and a longitudinal axis of the prosthetic heart valve (and/or with an outer surface of the frame 510c). The angle α1 may be larger than angle α2, which may be larger than angle α3. In some examples, angle α1 may be between about 35 degrees and 55 degrees, including about 45 degrees. In some examples, angle α2 may be between about 20 degrees and about 40 degrees, including about 30 degrees. In some examples, angle α3 may be between about 5 degrees and 25 degrees, including about 15 degrees. It should be understood that the angle values shown and described in connection with FIGS. 9A-9C are merely exemplary, and other angles may be suitable. In some examples, a single prosthetic heart valve having two or more engagement arms may have engagement arms that, in the absence of applied forces, form different angles with the longitudinal axis of the prosthetic heart valve and/or the outer surface of the frame thereof. In use, a prosthetic heart valve having anchor arms that are the same size (e.g. similar to prosthetic heart valve 100 or 300), or a prosthetic heart valve having anchor arms that are grouped in two or more different sizes (e.g. similar to prosthetic heart valve 200), may incorporate two or more groups of anchor arms with different angles in the absence of applied forces. For example, prosthetic heart valve 100 or 300 may be provided with a first group of three anchor arms 513a, a second group of three anchor arms 513b, and a third group of three anchor arms 513c, which may be provided in alternating positions around the circumference of the prosthesis. In some examples, the engagement arms 513c may tend to revert back toward their set shape more quickly, as spring tension may be higher when the engagement arm is inverted, compared to engagement 513b (which may, in turn, revert back toward its set shape more quickly due to higher spring forces when inverted than engagement arm 513a). It should be understood that anchor arms with only two different angles, or more than three different angles, may be used. It should also be understood that the different angles may be applied to anchor arms having different lengths, for example longer anchor arms having a smaller angle and shorter anchor arms having a larger angle (or vice versa). In some examples, the different angles illustrated and described in FIGS. 9A-9C may be achieved by shape-setting (e.g. via heat treatment) the engagement arms at different positions. Further, it should be understood that the anchor arms 513a-513c, despite having different set angles, may all follow a path that is on a line that intersects with the central longitudinal axis of the prosthetic heart valve. However, in other examples (see FIGS. 10A-10C and description below), the engagement arms may be angled rotationally (e.g. to point in a direction that does not intersect with the longitudinal axis of the prosthetic heart valve).

FIGS. 10A-10C illustrate more examples of prosthetic heart valves, with FIGS. 10A-10C showing portions of frames 610a-610c from a front view. The prosthetic heart valves shown in FIGS. 10A-10C may be substantially similar or identical to the other prosthetic heart valves described herein, with the primary difference of the embodiments of FIGS. 10A-10C being the different set angles β1, β2, β3 of the engagement arms 613a-613c. The views of FIGS. 10A-10C illustrate a portion of the frame 610a-610c when in an expanded or deployed condition, or otherwise in the absence of applied forces. It should be understood that the prosthetic heart valves represented in FIGS. 10A-10C may include any number of engagement arms 613a-613c, similar to other embodiments described herein. As shown in FIG. 10A, in the absence of applied forces, engagement arm 613a forms an angle of β1 between the engagement arm 613a and a longitudinal axis of the prosthetic heart valve. In particular, β1 forms a rotational angle of approximately 0 degrees. In other words, although the engagement arm 613a may have an angle similar to any of those shown in FIGS. 9A-9C, when viewed head-on, the engagement arm 613a forms a 0 degree rotational angle with longitudinal axis L. In this respect, the embodiment of FIG. 10A may represent any of the embodiments of FIGS. 9A-9C, and is shown largely to provide better context for the embodiments of FIGS. 10B-10C. As shown in FIG. 10B, in the absence of applied forces, engagement arm 613b forms a rotational angle of β2 between the engagement arm 613b and the longitudinal axis L of the prosthetic heart valve. In other words, although engagement arm 613b may have any angle α1, α2, or α3 (or any other angle) shown and described in connection with FIGS. 9A-9C, engagement arm 613b has an additional angle β2 relative to the longitudinal axis L, such that the engagement arm 613b sweeps in a rotational or clockwise angle (although a counterclockwise angle could be provide instead). With this configuration, a line passing through engagement arm 613b does not intersect the longitudinal axis L of the prosthetic heart valve. In the illustrated example, the rotational angle β2 may be between about 20 degrees and about 40 degrees, including about 30 degrees (although similar negative angles, which may simply be angles in the opposite direction as shown in FIG. 10B, may be used in the alternative). As shown in FIG. 10C, in the absence of applied forces, engagement arm 613c forms a rotational angle of β3 between the engagement arm 613c and the longitudinal axis L of the prosthetic heart valve. In other words, although engagement arm 613c may have any angle α1, α2, or α3 (or any other angle) shown and described in connection with FIGS. 9A-9C, engagement arm 613c has an additional angle β3 relative to the longitudinal axis L, such that the engagement arm 613c sweeps in a rotational or clockwise angle (although a counterclockwise angle could be provide instead). With this configuration, a line passing through engagement arm 613c does not intersect the longitudinal axis L of the prosthetic heart valve. In the illustrated example, the rotational angle β3 may be between about 30 degrees and about 50 degrees, including about 45 degrees (although similar negative angles, which may simply be angles in the opposite direction as shown in FIG. 10C, may be used in the alternative). The angles β1 through β3 may also be defined by viewing the prosthetic heart valve from a front view, and projecting the engagement arm onto a two-dimensional plane bisecting the prosthetic heart view, with that plane orthogonal to the viewer, and determining the angle between the engagement arm and the longitudinal axis of the prosthetic heart valve. It should be understood that the angle values shown and described in connection with FIGS. 10A-10C are merely exemplary, and other angles may be suitable. In some examples, a single prosthetic heart valve having two or more engagement arms may have engagement arms that, in the absence of applied forces, form different rotational or sweeping angles with the longitudinal axis of the prosthetic heart valve. In use, a prosthetic heart valve having anchor arms that are the same size (e.g. similar to prosthetic heart valve 100 or 300), or a prosthetic heart valve having anchor arms that are grouped in two or more different sizes (e.g. similar to prosthetic heart valve 200), may incorporate two or more groups of anchor arms with different rotational or sweeping angles in the absence of applied forces. For example, prosthetic heart valve 100 or 300 may be provided with a first group of three anchor arms 613a, a second group of three anchor arms 613b, and a third group of three anchor arms 613c, which may be provided in alternating positions around the circumference of the prosthesis. It should be understood that anchor arms with only two different rotational angles, or more than three different rotational angles, may be used. It should also be understood that the different rotational or sweeping angles may be applied to anchor arms having different lengths, for example longer anchor arms having a smaller angle and shorter anchor arms having a larger angle (or vice versa). In some examples, the different angles illustrated and described in FIGS. 10A-10C may be achieved by shape-setting (e.g. via heat treatment) the engagement arms at different positions. Further, it should be understood that the rotational or sweeping angles shown and described in connection with FIGS. 10A-10C may be combined with the angles shown and described in connection with FIGS. 9A-9C in any desired combination. Finally, it should be understood that the different angled arms of FIGS. 9A-9C and /or 10A-10C may be combined with other features described herein, such as engagement arms having different lengths and/or different spring forces and/or echogenic properties. Having the engagement arms at non-uniform angles may allow for the engagement arms to behave non-uniformly, which may help to avoid the need to simultaneously position a plurality of anchor arms at the same time that all behave substantially identically.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system comprising:
a prosthetic atrioventricular valve for implantation into a native heart valve, the prosthetic atrioventricular valve including:
a collapsible and expandable frame extending in a longitudinal direction from an inflow end of the frame toward an outflow end of the frame;
a plurality of prosthetic leaflets mounted within the frame and configured to allow blood to flow through the prosthetic heart valve in an antegrade direction from the inflow end of the frame toward the outflow end of the frame, but to prevent blood from flowing through the prosthetic heart valve in a retrograde direction from the outflow end of the frame toward the inflow end of the frame; and
a plurality of anchor arms, each anchor arm having (i) a main body coupled to the frame and (ii) a tip at a free end of the anchor arm, the tip of the anchor arm having a width that is larger than a width of the main body; and
a delivery device including a catheter configured to receive the prosthetic atrioventricular valve therein while the prosthetic atrioventricular valve is in a collapsed condition,
wherein when the prosthetic atrioventricular valve is in an expanded condition, the tip of each of the plurality of anchor arms points toward the inflow end of the frame and is spaced radially outward of the frame to define a leaflet-receiving space,
wherein when the prosthetic atrioventricular valve is in the collapsed condition within the catheter, the tip of each of the plurality of anchor arms points away from the inflow end of the frame, and the tips of at least two of the plurality of anchor arms are positioned at different axial positions relative to the catheter.

2. The system of claim 1, wherein when the prosthetic atrioventricular valve is in the collapsed condition within the catheter, no two circumferentially adjacent ones of the tips of the plurality of anchor arms are positioned at a same axial position relative to the catheter.

3. The system of claim 1, wherein the plurality of anchor arms includes nine anchor arms arranged in a first group of three anchor arms, a second group of three anchor arms, and a third group of three anchor arms, such that when the prosthetic atrioventricular valve is in the collapsed condition within the catheter, the tips of the three anchor arms in the first group are positioned at a first axial position relative to the catheter, the tips of the three anchor arms in the second group are positioned at a second axial position relative to the catheter, and the tips of the three anchor arms in the third group are positioned at a third axial position relative to the catheter, the first axial position being different than the second axial position, the second axial position being different than the third axial position, and the third axial position being different than the first axial position.

4. The system of claim 3, wherein (i) each anchor arm in the first group is positioned between one of the anchor arms in the second group and one of the anchor arms in the third group; (ii) each anchor arm in the second group is positioned between one of the anchor arms in the first group and one of the anchor arms in the third group; and (iii) each anchor arm in the third group is positioned between one of the anchor arms in the first group and one of the anchor arms in the second group.

5. The system of claim 3, wherein upon deployment of the prosthetic atrioventricular valve from the delivery device, the three anchor arms in the first group are configured to exit the delivery device before the three anchor arms in the second group exit the delivery device.

6. The system of claim 5, wherein upon deployment of the prosthetic atrioventricular valve from the delivery device, the three anchor arms in the second group are configured to exit the delivery device before the three anchor arms in the third group.

7. The system of claim 1, wherein the tips of the plurality of anchor arms are formed with echogenic material.

8. A system comprising:
a prosthetic atrioventricular valve for implantation into a native heart valve, the prosthetic atrioventricular valve including:
a collapsible and expandable frame extending in a longitudinal direction from an inflow end of the frame toward an outflow end of the frame;
a plurality of prosthetic leaflets mounted within the frame and configure to allow blood to flow through the prosthetic heart valve in an antegrade direction from the inflow end of the frame toward the outflow end of the frame, but to prevent blood from flowing through the prosthetic heart valve in a retrograde direction from the outflow end of the frame toward the inflow end of the frame; and
a plurality of anchor arms, each anchor arm having (i) a main body coupled to the frame and (ii) a tip at a free end of the anchor arm; and
a delivery device including a catheter configured to receive the prosthetic atrioventricular valve therein while the prosthetic atrioventricular valve is in a collapsed condition,
wherein when the prosthetic atrioventricular valve is in an expanded condition, the tip of each of the plurality of anchor arms points toward the inflow end of the frame and is spaced radially outward of the frame to define a leaflet-receiving space,
wherein when the prosthetic atrioventricular valve is in the collapsed condition within the catheter, the tip of each of the plurality of anchor arms points away from the inflow end of the frame, and the tips of at least two of the plurality of anchor arms are positioned at different axial positions relative to the catheter,
wherein the tips of the plurality of anchor arms are formed with echogenic material.

9. The system of claim 8, wherein the echogenic material is a biocompatible metal or metal alloy.

10. The system of claim 8, wherein at least some of the plurality of anchor arms include a frictional element on the main body adjacent to the tip.

11. The system of claim 10, wherein the frictional element is selected from the group consisting of: spikes, hooks, tines, or barbs.

12. The system of claim 8, wherein when the prosthetic atrioventricular valve is in the collapsed condition within the catheter, the tips of at least two of the plurality of anchor arms are positioned at different axial positions relative to the catheter.

13. A method of implanting a prosthetic atrioventricular valve into a native atrioventricular valve of a patient, the method comprising:
positioning the prosthetic atrioventricular valve in a collapsed condition within a catheter of a delivery device, the prosthetic atrioventricular valve including a collapsible and expandable frame extending in a longitudinal direction from an inflow end of the frame toward an outflow end of the frame, a plurality of prosthetic leaflets mounted within the frame, and a plurality of anchor arms, each anchor arm having (i) a main body coupled to the frame and (ii) a tip at a free end of the anchor arm, the tip of the anchor arm having a width that is larger than a width of the main body;
advancing the catheter through vasculature of the patient until the catheter is positioned in or adjacent to the native atrioventricular valve, wherein during the advancing, the tip of each of the plurality of anchor arms points away from the inflow end of the frame, and the tips of at least two of the plurality of anchor arms are positioned at different axial positions relative to the catheter;
when the catheter is positioned in or adjacent to the native atrioventricular valve, withdrawing the catheter relative to the prosthetic atrioventricular valve so that a tip of a first one of the plurality of anchor arms exits the catheter before a tip of a second one of the plurality of anchor arms exits the catheter;
allowing at least one of the anchor arms to start to transition to a set shape in which the tip of the at least one of the anchor arms points toward the inflow end of the frame after the at least one of the anchor arms exits the catheter, so that the at least one of the anchor arms hooks behind a native leaflet of the native atrioventricular valve; and
after the at least one of the anchor arms hooks behind a native leaflet of the native atrioventricular valve, fully deploying the prosthetic atrioventricular valve from the catheter and allowing the prosthetic atrioventricular valve to self-expand into the native atrioventricular valve.

14. The method of claim 13, wherein when the prosthetic atrioventricular valve is in the collapsed condition within the catheter, no two circumferentially adjacent ones of the tips of the plurality of anchor arms are positioned at a same axial position relative to the catheter.

15. The method of claim 13, wherein the plurality of anchor arms includes nine anchor arms arranged in a first group of three anchor arms, a second group of three anchor arms, and a third group of three anchor arms, such that when the prosthetic atrioventricular valve is in the collapsed condition within the catheter, the tips of the three anchor arms in the first group are positioned at a first axial position relative to the catheter, the tips of the three anchor arms in the second group are positioned at a second axial position relative to the catheter, and the tips of the three anchor arms in the third group are positioned at a third axial position relative to the catheter, the first axial position being different than the second axial position, the second axial position being different than the third axial position, and the third axial position being different than the first axial position.

16. The method of claim 15, wherein (i) each anchor arm in the first group is positioned between one of the anchor arms in the second group and one of the anchor arms in the third group; (ii) each anchor arm in the second group is positioned between one of the anchor arms in the first group and one of the anchor arms in the third group; and (iii) each anchor arm in the third group is positioned between one of the anchor arms in the first group and one of the anchor arms in the second group.

17. The method of claim 15, wherein the first one of the plurality of anchor arms is part of the three anchor arms of the first group, and the second one of the plurality of anchor arms is part of the three anchor arms of the second group.

18. The method of claim 17, wherein, while withdrawing the catheter relative to the prosthetic atrioventricular valve, the three anchor arms of the first group exit the catheter before the three anchor arms of the second group exit the catheter, and the three anchor arms of the second group exit the catheter before the three anchor arms of the third group exit the catheter.

19. The method of claim 13, wherein the tips of the plurality of anchor arms are formed with echogenic material.

20. The method of claim 19, further comprising performing ultrasound imaging to determine positions of the tips of the plurality of anchor arms.
